(19) United States
(12) Patent Application Publication
(10) Pub. No.: US 2025/0354985 A1
(43) Pub. Date: Nov. 20, 2025

(54) APPARATUS FOR PERFORMING IMMUNOMETRIC TESTS

(71) Applicant: DIESSE DIAGNOSTICA SENESE S.P.A., Milano (IT)

(72) Inventors: Alessandra BROGI, Monteriggioni (SI) (IT); Barbara FERRISI, Monteriggioni (SI) (IT); Michele MELONI, Monteriggioni (SI) (IT)

(73) Assignee: DIESSE DIAGNOSTICA SENESE S.P.A., Milano (IT)

(21) Appl. No.: 19/108,349

(22) PCT Filed: Sep. 21, 2023
(86) PCT No.: PCT/EP2023/076094

§ 371 Date: Mar. 3, 2025

(30) Foreign Application Priority Data
Sep. 23, 2022 (IT) 102022000019581

(51) Int. Cl. G01N 33/543; G01N 21/76; G01N 35/00

(52) U.S. Cl. CPC G01N 33/54366; G01N 21/76; G01N 35/0099

(57) ABSTRACT

An apparatus for performing immunometric tests is disclosed. The apparatus has a housing element configured to house a plurality of single sample test devices and includes housings for a solid phase, a sample to be analyzed, and reagents, means for acting on the housing element to allow the execution of the desired immunometric test, and a control unit for managing the apparatus. The apparatus includes means for the automatic execution of different immunometric tests, including a detector for executing Enzyme-Linked Immunosorbent Assay (ELISA) tests, a detector for executing Chemiluminescent Immunoassay (CLIA) tests; and an image detector in communication with the control unit for acquiring and processing images of the sample.

### FIELD OF APPLICATION

The present invention refers to an apparatus for performing immunometric tests, in particular using ready-to-use diagnostic devices for a single determination (“monotest device or single sample test device”), for example for the identification of infectious diseases, autoimmunity, tumor markers, allergies, inflammations, analysis of bone metabolism, and the like. The following description refers to this field of application with the only purpose to simplify its exposition.

### PRIOR ART

As known, immunometry takes advantage of the antigen-antibody reaction to assess the concentration of a given analyte in a biological sample to be analyzed. To identify a specific antibody, the antigen against which said antibody is directed is used, since, if said antibody is present, it will bind said antigen; alternatively, in a specular way, a specific antibody may be used to detect an antigen. This interchangeability of antigens and antibodies as ligands and as detecting agents is at the basis of the great versatility of the immunometric tests.

The presence of the antigen-antibody complex thus formed, which can be observed by means of specific procedures, is a sign of the presence of the antibody (or of the antigen) that is sought. Thus, immunometric tests allow to detect specific antigens or antibodies in the blood and in other body fluids, and are therefore useful for the identification of infectious diseases and other pathological conditions.

As mentioned above, in accordance with known methodologies, the above-mentioned immune reaction is used together with enzymatic reactions to produce a colored signal that can be easily measured in a quantitative way with appropriate detectors.

Nowadays, there are apparatuses that use diagnostic devices (having the solid phase and the reagents) for a single determination and that are capable of performing immunometric tests in a semi-automatic way; however, said apparatuses are generally limited to a single type of test (for example, only to the ELISA test). Instead, other types of known apparatuses are too complex and do not allow the easy and rapid use thereof.

The technical problem of the present invention is to provide an apparatus having structural and functional features capable of overcoming the limitations and the drawbacks of the prior art, in particular which has a simple structure and use, and at the same time which is versatile and usable for different immunometric tests.

### SUMMARY OF THE INVENTION

The solution idea underlying the present invention is to integrate different instruments in a single apparatus (that uses ready-to-use test devices for a single sample test), each instrument being configured for the automatic execution of a specific type of test, wherein said instruments act on samples transferred in an automatic way and can act simultaneously. In particular, the apparatus is equipped with readers for carrying out tests based on ELISA, CLIA and Macro Array methodology, thereby proving a great versatility of use.

Based on said solution idea, the above-mentioned technical problem is solved by an apparatus for performing immunometric tests, comprising a housing element configured to house at least one (preferably a plurality of) test device which is of the single use-type for a single sample test (single determination), and which comprises a plurality of housings at least for a solid phase, for a sample to be analyzed, and for reagents, means apt to act on the housing element to allow the execution of the desired immunometric test, and a control unit apt to manage the apparatus, characterized in that it comprises at least the following means for the automatic execution of different immunometric tests:
at least one optical detector configured to acquire signals and being in operative communication with the control unit which is configured to process said signals for the execution of tests of the Enzyme-Linked Immunosorbent Assay (ELISA) type;
at least one optical detector configured to acquire signals and being in operative communication with the control unit which is configured to process said signals for the execution of tests of the Chemiluminescent Immunoassay (CLIA) type; and
at least one image detector for acquiring images of the test device (containing the sample transferred thereinto), said image detector being in communication with the control unit, which is configured to process said images.

According to the present invention, the apparatus is configured to receive, for each of said tests, the test devices which comprise a body which houses, along the longitudinal axis thereof, at least one reaction well containing the solid phase from a microtitration plate, at least one recess for the sample to be analyzed, and one or more recesses for respective one or more reagents.

The test device may therefore be considered as part of the apparatus of the present invention. Therefore, advantageously according to the present invention, the apparatus further comprises at least one test device, which comprises a body which houses, along a longitudinal axis thereof, the housings, wherein said housings comprise at least one reaction well containing the solid phase from a microtitration plate, at least one recess for the sample to be analyzed, and one or more recesses for respective one or more reagents.

More particularly, the invention comprises the following additional and optional features, taken individually or in combination if necessary.

According to an aspect of the present invention, the apparatus may also comprise another housing element adapted to house the samples to be analyzed, arranged in any suitable way.

According to an aspect of the present invention, the control unit may be configured to perform said at least three immunometric tests individually or even simultaneously.

According to an aspect of the present invention, based on the images of the image detector, the control unit may be configured to perform macroarray tests, the control unit being configured to process the images by means of machine learning techniques.

According to an aspect of the present invention, the detector apt to perform the ELISA test may be a photometer/spectrophotometer.

According to an aspect of the present invention, the detector apt to perform the CLIA test may be a photomultiplier.

According to an aspect of the present invention, the apparatus may further comprise a housing area for test tubes containing the sample to be analyzed, and a mechanical arm configured to take the test tubes from the housing area and to perform the automatic transfer thereof into the test devices housed in the housing element.

According to an aspect of the present invention, the housing element may comprise a first rotor or inner rotor and a second rotor or outer rotor, which are arranged concentric one inside the other, wherein the second rotor is external to the first rotor and is a pre-cycle support adapted to house the test devices coming from a loading area, and wherein the first rotor is configured to receive the test devices from the second rotor and is the support in communication with the means for the execution of the reaction and of the desired test. This very advantageous aspect may also be implemented regardless of the number and type of tests that are integrated in the apparatus, and is a general feature of the invention combinable with the preamble of claim 1 and with any other optional aspect herein described.

According to an aspect of the present invention, the first rotor and the second rotor may be moved by movement means which are independent from each other and controlled in an automated way.

According to an aspect of the present invention, the second rotor may be configured to perform a pre-cycle in which the sample in the test tube is transferred into the appropriate housing of the test device, and the first rotor may be configured to perform the shaking of the test device containing the sample and reagents.

According to an aspect of the present invention, the apparatus may comprise one or more needles for transferring the samples and reagents in the pre-cycle step in the second rotor and for carrying out the test in the first rotor.

The features and advantages of the apparatus according to the present invention will become apparent from the following description of an exemplary embodiment thereof, given by way of non-limiting example with reference to the accompanying drawings.

### BRIEF DESCRIPTION OF THE FIGURES

In the figures:

FIG. 1 is a general outline of the apparatus according to the present invention;

FIG. 2 is a perspective view of the apparatus according to an embodiment of the present invention;

FIGS. 3A and 3B relate to test devices used by the apparatus according to embodiments of the present invention;

FIG. 4 is a top view of the apparatus of FIG. 2; and

FIGS. 5, 6 and 7 show details of the apparatus according to embodiments of the present invention.

### DETAILED DESCRIPTION

With reference to those figures, an apparatus for performing immunometric tests according to the present invention is globally and schematically indicated with the reference number 1.

It is worth noting that the figures represent schematic views and are not necessarily drawn to scale, but instead they are drawn so as to emphasize the important features of the invention. Further, in the figures, the different elements are shown in a schematic way, their shape being variable depending on the desired application. It is further worth noting that in the figures identical reference numerals refer to identical elements in shape or function. Finally, particular features described in relation to an embodiment illustrated in a figure are also applicable to other embodiments illustrated in the other figures.

It is also noted that, unless the opposite is expressly indicated, the described process steps may also be inverted if necessary.

The present description relates to a very versatile apparatus 1 for performing various immunometric tests, said apparatus 1 being configured to receive and use diagnostic devices (hereinafter also called “test devices” and being indicated with reference D), which are ready-to-use and for a single determination (i.e. they monotest devices; in other words, they are devices for a single sample test).

For the purpose of allowing the execution of the operations described below, the apparatus 1 comprises a control unit (identified with reference C), including suitable memory units MEM and suitably programmed and designated for managing and automatically controlling the apparatus and for the analysis of the data of measurement. The control unit C may be, for example, a computerized unit integrated in the apparatus 1. Moreover, the control unit C may be a single unit or may comprise a plurality of local and/or remote units, possibly communicating with each other and each being designated for performing specific operations. The control unit C is thus apt to control the apparatus 1 to obtain the desired functionalities. In any case, the present invention is not limited in any way by the architecture used for the control unit C, which may be in general any suitable computerized unit, comprising one or more unit(s) based on the needs and/or circumstances.

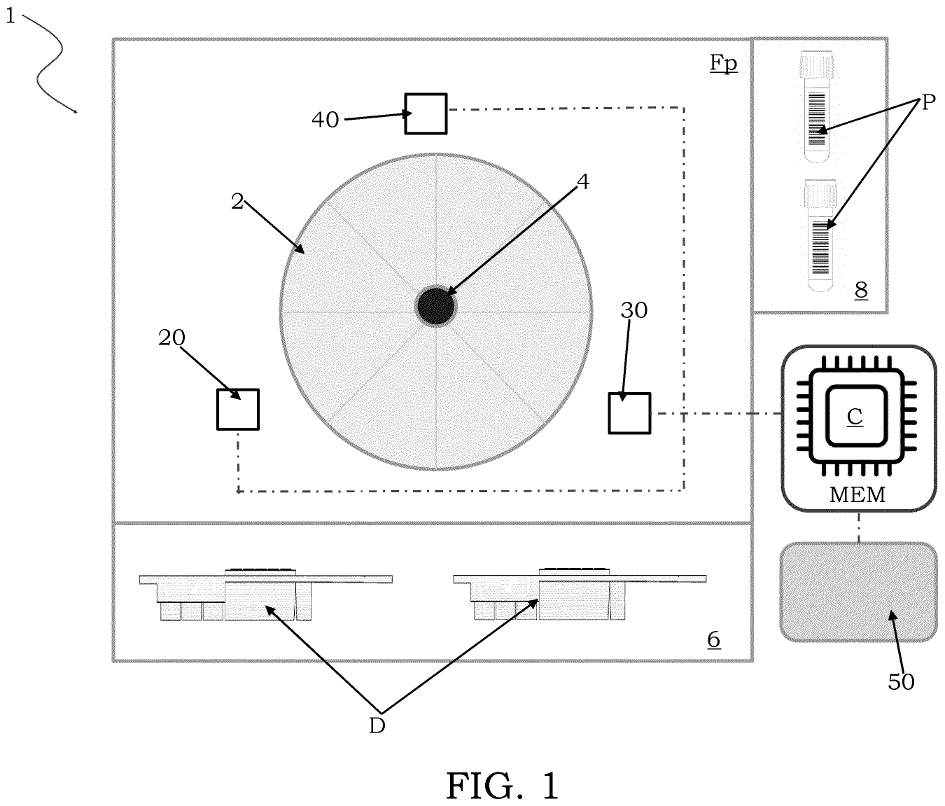

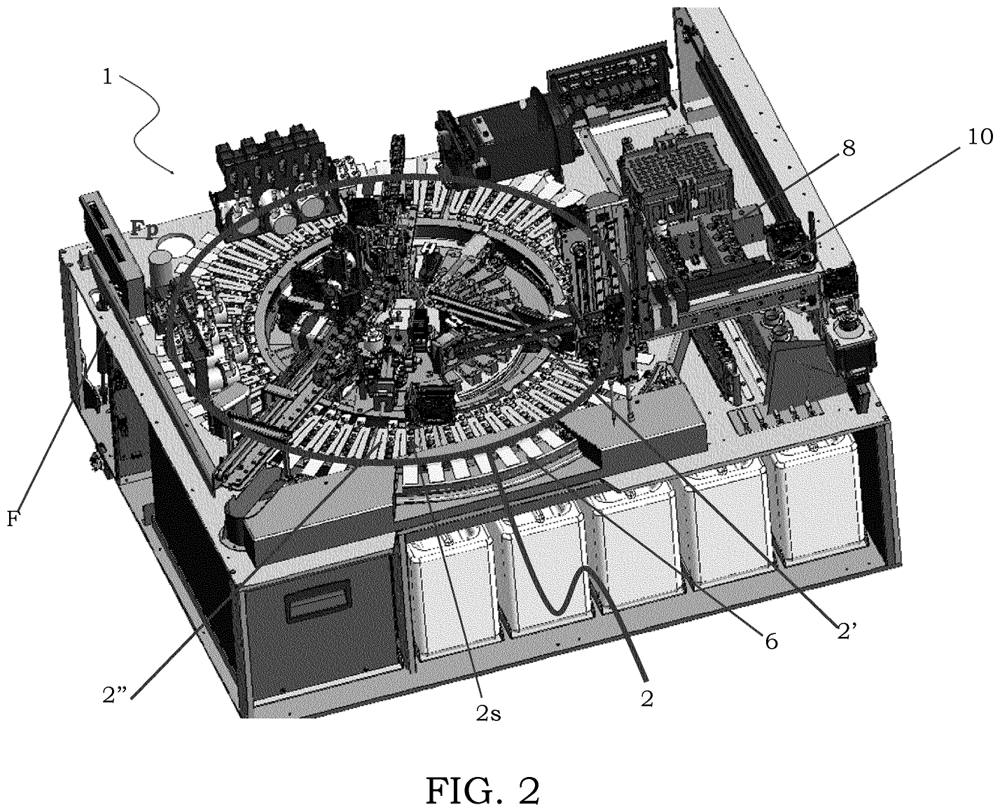

As illustrated in FIG. 1 (which represents a general, intentionally schematic view) and in FIG. 2, the apparatus 1 comprises a main frame F providing support to all its components. Obviously, the apparatus 1 is not limited to the configuration of the main frame F, as well as to the shape of possible outer covering casings (not shown in the figures). As illustrated, the main frame F defines a working surface Fp which is substantially horizontal (i.e. parallel to the support plane) and on which the main measurement components are arranged. FIG. 2 illustrates thus the components that are generally covered by a not-shown covering element.

The apparatus 1 further comprises a housing element (indicated with the reference number 2) for a plurality of test devices D, which are housed in appropriate housing seats 2s, as illustrated in the non-limiting example of FIG. 2.

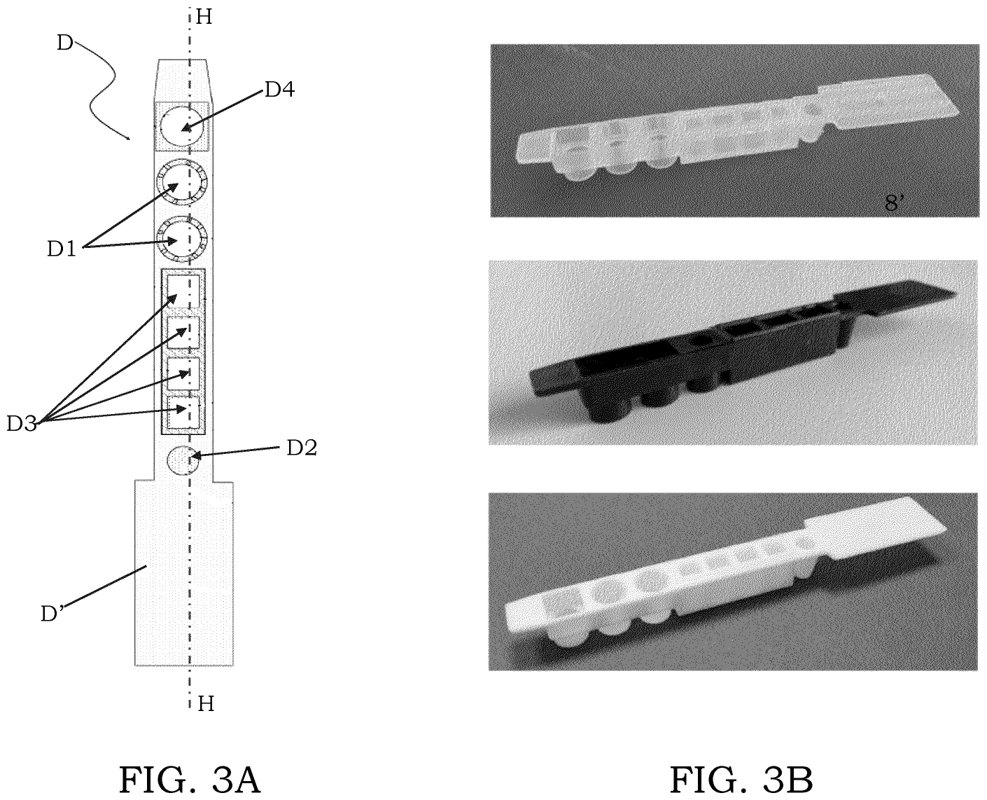

As previously mentioned, and as shown in detail in FIGS. 3A and 3B, the test devices D are configured to be of the single use-type (monotest devices) and comprise housings at least for a solid phase, for a sample to be analyzed, and for reagents (there are generally various housings for various reagents). More in particular, the apparatus 1 is configured to receive said test devices D comprising a body D′ which houses, along the longitudinal axis H-H thereof, one or more wells D1 (reaction wells) with reaction elements from a microtitration plate (solid phase), at least one recess D2 for the sample to be analyzed, and one or more recesses D3 for the reagents. In the shown example, there are two wells D1 so that it is possible to perform two determinations on the same sample. Optionally, there is an end recess D4 for execution of the substrate blank. The material of the test device D is not limited to a particular type and may be any plastic material suitable for the purpose. As will be discussed below and as shown in FIG. 3B, depending on the type of test performed, a particular test device D is selected, which is characterized by a given surface opacity and/or coloration, although the outer structure is substantially identical for each determination.

For example, the solid phase may comprise purified native proteins or recombinant proteins that are printed on the bottom of a microtitration plate, which is then subdivided into the single reaction wells which are individually inserted into said test device D.

Generally, the test device D comprises, along the longitudinal axis thereof, at least one recess to receive the reaction well from a preexisting microtitration plate, thereby receiving the so-called solid phase which is at the bottom of the well.

In the context of the present disclosure, the solid phase thus corresponds to the bottom of the reaction well of the test device, which comes from the subdivision of the existing microtitration plate (in other words, the reaction well thus contains the solid phase at the bottom thereof, said well resulting from the subdivision of the original microtitration plate).

This is very advantageous since this simple technique allows to perform one or more tests on a single sample without being bound to a plate with a plurality of wells, for example ninety-six wells.

In accordance with embodiments of the present invention, for example in the case of the macroarray test, the solid phase of the well is the bottom of the well to which a coating of biomarker-specific antigen protein is applied.

The apparatus 1 comprises means (indicated in FIG. 1 in a general and intentionally schematic manner with the reference number 4) apt to act on the housing element 2 in order to allow the execution of the desired immunometric test. The means 4 may comprise various components, including movement means (for example suitable motors) for moving the housing element 2, in particular for rotating it, and means for shaking the test devices D, as will be detailed hereinafter.

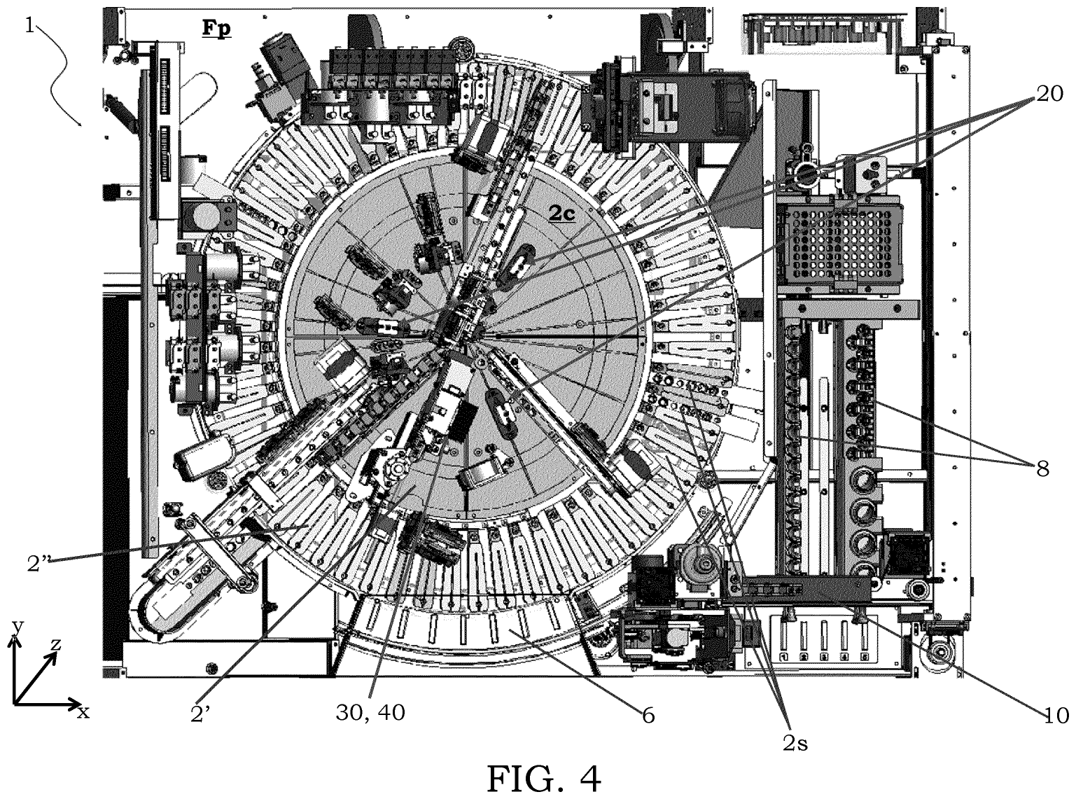

Even more in particular, as also shown in FIG. 4, the housing element 2 is structured so as to comprise a first rotor or inner rotor 2′ and a second rotor or outer rotor 2″, which are arranged concentric one inside the other, in particular the inner rotor 2′ is arranged inside the outer rotor 2″. In the context of the present description, the term “rotor” indicates a support, not limited to a particular shape, capable of rotating around a prefixed axis, thereby causing the rotation of the various test devices D housed therein. FIG. 4 (as well as the subsequent figures) also shows an optional covering element 2c of the rotor, which, instead, was not illustrated in FIG. 1 in order to show the various details.

The outer rotor 2″ may house a high number of test devices D, for example, it may comprise more than sixty housing seats 2s; in an example, it comprises fifty-six housing seats 2s, without, however, being limited to this number. The rotation of this outer rotor 2″ may occur by means of an helicoidal-teeth gear that engages with a corresponding pinion mounted on a stepper motor provided with an encoder; a sensor for controlling the zero position is also provided. Obviously, other suitable configurations are also within the scope of the present invention, which is not limited to the examples mentioned herein.

Specifically, the outer rotor 2″ is a pre-cycle support adapted to house the test devices D coming from a loading area (indicated with the reference number 6 and in which the operator manually inserts the test devices D into appropriate seats). In particular, this outer rotor 2″ is configured to perform a pre-cycle in which the sample, which is initially contained in a test tube P (FIG. 1), is transferred into the appropriate housing D2 of the test device D (FIG. 3A).

As mentioned, the loading of the test devices D on the outer rotor 2″ may occur manually, for example through a controlled-access front door. The opening of this door allows to load on the outer rotor 2″ a limited number of test devices D; further test devices D may then be loaded after the rotation of the outer rotor 2″. Positioning sensors and barcode readers for the recognition of the test devices D fed into the outer rotor 2″ are also provided.

As regards the inner rotor 2′, it is configured to receive the test devices D from the outer rotor 2″, and it is the support on which the reaction, and thus the desired test, is actually performed (for this reason, it is also indicated as “reaction rotor”). The inner rotor 2′ is preferably configured to house a smaller number of test devices D compared to the outer rotor 2″, for example, it may comprise up to thirty housing seats, without, however, being limited to a specific number.

The inner rotor 2′ is thus in communication with the means apt for the actual execution of the test (for example, the shaking means, which are part of the above-mentioned means 4), which may be considered an integral part thereof. In other words, the inner rotor 2′ is thus provided with means that allow the shaking of the test devices D during the reaction, and it is therefore configured to perform the shaking of the test device D and to allow the execution of the desired analysis.

In an embodiment, the inner rotor 2′ is arranged in a chamber that is thermally controlled, for example by a Peltier module.

Further, in an embodiment, similarly to what was mentioned above for the outer rotor 2″, the rotation of the inner rotor 2′ occurs by means of an helicoidal-teeth gear in engagement with a corresponding pinion mounted on a stepper motor provided with an encoder, for example mounted on the outer perimeter of the rotor.

The rotors 2′ and 2″ may thus be moved by movement means which are independent from each other and controlled in an automated way.

In this way, the main structure of the apparatus 1 is based on two concentric rotors: the outer rotor 2″ which is intended for loading the test devices D and for the pre-cycle step, and the inner rotor 2′ onto which the test devices D are subsequently transferred for the execution of the reaction and thus of the test.

Between the inner rotor 2′ and the outer rotor 2″ there is an assembly configured to automatically transfer the test devices D from one rotor to the other one, this assembly comprising for example a linear actuator and suitable sensors. Between the two rotors 2′ and 2″ there are also two forks, one closer to the outer rotor 2″ and the other one closer to the inner rotor 2′, with the purpose of detecting the completed passage of the test device D between the two rotors.

Further, there is an assembly configured to automatically expel the used devices from the inner rotor 2′ toward the outer rotor 2″ at the end of the test, for example by means of linear actuators and sensors, in particular by passing through housing seats of the outer rotor 2″ that are expressly dedicated to said purpose. For example, four housing seats to be used only for discharging and not for loading the test devices D may be provided (but the number is not limited to four), the movement of the rotors being thus suitably controlled and synchronized by the control unit C to allow the above-mentioned discharging operations through said dedicated housing seats. The used test devices D are then discharged into appropriate containers, which are for example arranged under the working surface Fp, as illustrated in FIG. 2, said containers being provided with sensors for the detection of the fill level.

As previously mentioned, there is a housing area 8 for the loading of racks that contain the samples to be processed, said samples being contained in the test tube P, for example on the side of rotors 2′ and 2″. In particular, in an embodiment, once the test tubes P are arranged in the housing area 8 inside the various racks, there is a mechanical arm 10 configured to take said test tubes P from the housing area 8 and to perform the automatic transfer thereof into the test devices D housed in the housing element 2, in particular in the outer rotor 2″. Also in this case, there are barcode readers for reading the codes of the racks and of the test tubes P, thus identifying the various racks and distinguishing the empty positions of the rack from the full ones, i.e. those positions in which the bottom code of the rack has been detected, which indicates the absence of the test tube.

The advantage of the above-mentioned two-rotor configuration is to allow a continuous loading by means of the outer rotor 2″ without interrupting the test performed in the inner rotor 2′ and without the need to distinguish among the various types of test.

This two-rotor configuration is independent of the other features described herein, for example independent of the integration of different instruments for different tests. As a consequence, an apparatus with the features of the preamble of the attached independent claim and with the housing element comprising the double rotor (as in claim 7) may be defined, independently of the type and number of tests that can be performed, the presence of means for the execution of at least one test being sufficient. All the other optional combinations are valid also in this case and are optional.

Once defined the above-mentioned mechanical structure of the apparatus 1, it is desirable to perform, with said apparatus 1, different tests without the need to change machine, by simply selecting the desired test.

Advantageously according to the present invention, the apparatus 1 comprises at least the following means for the automatic execution of different immunometric tests:
at least one detector 20 configured to acquire signals from the sample (in particular from the reaction well) and being in operative communication with the control unit C which is configured to process said signals for the execution of tests of the Enzyme-Linked Immunosorbent Assay (ELISA) type;
at least one detector 30 configured to acquire signals from the sample (in particular from the reaction well) and being in operative communication with the control unit C which is configured to process said signals for the execution of tests of the Chemiluminescent Immunoassay (CLIA) type; and
at least one image detector 40 for acquiring images of the test device D housed in the inner rotor 2′, said image detector 40 being in communication with the control unit C, which is configured to process said images and to provide the desired measurement results. In particular, based on the images of the image detector 40, the control unit C is configured to perform macroarray tests, for example by processing the acquired images by means of machine learning techniques.

Conveniently, the control unit C is configured to perform the above-mentioned three immunometric tests individually or even simultaneously.

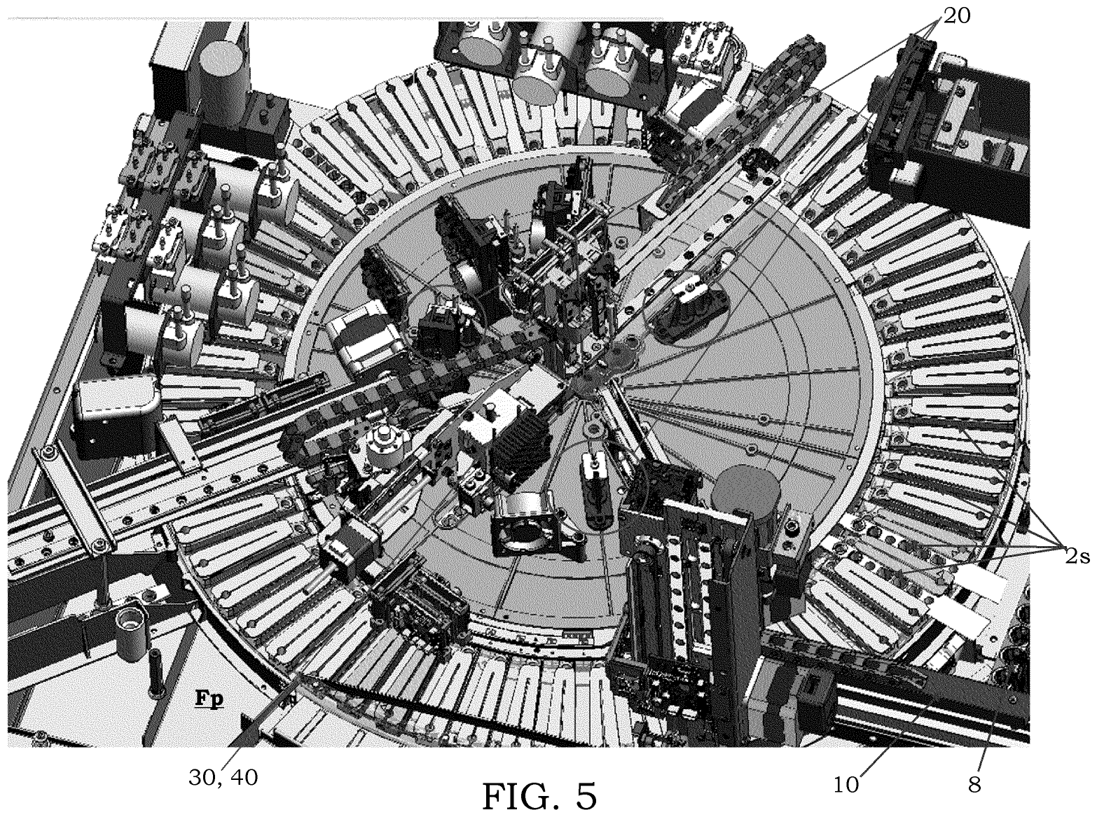

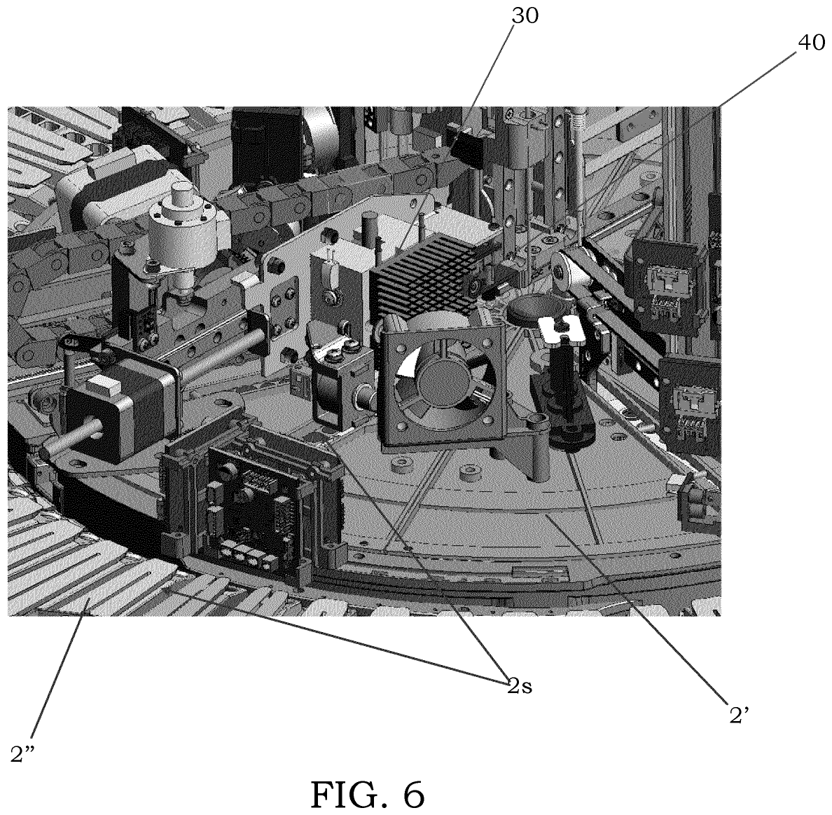

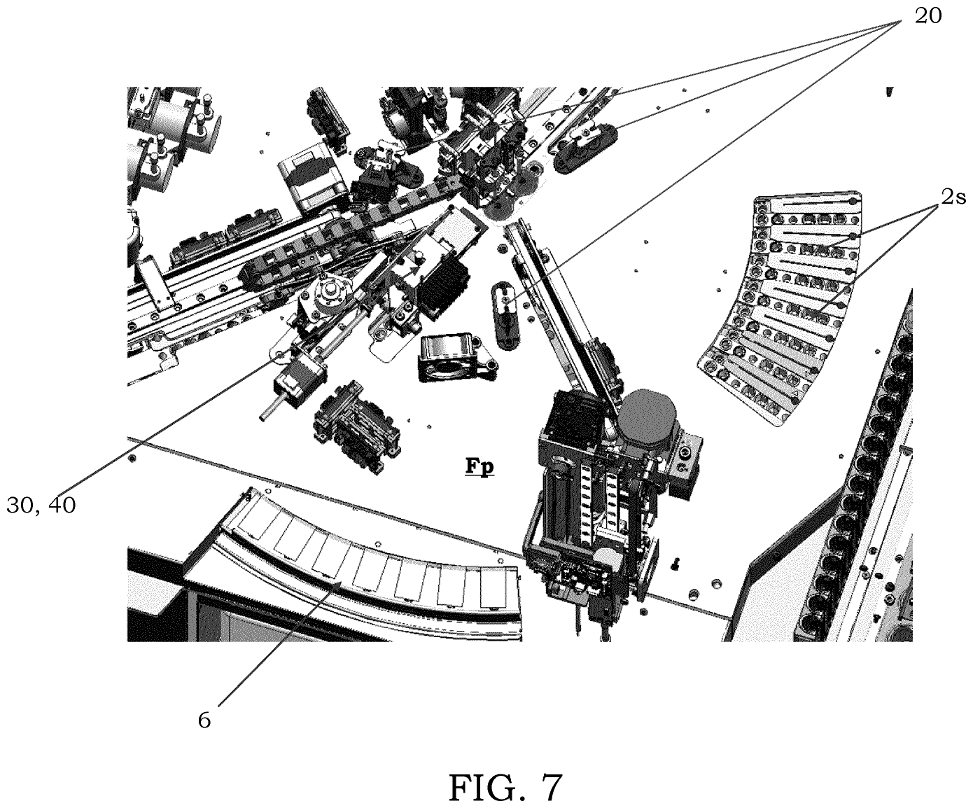

It is thus possible to perform, in the same cycle, tests that use the above-mentioned three different technologies (namely ELISA, CLIA and macroarray). The apparatus 1 is thus provided with various reading devices (comprising suitable sensors) configured to detect information relating to the samples in the test devices D, as detailed in FIGS. 5 to 7.

In particular, the detector 20, which is apt to perform the ELISA test, is a photometer/spectrophotometer. Even more in particular, it comprises suitable filters to perform the readings at certain wavelengths in the visible range, for example around 450 nm and 650 nm. These filters may be positioned on a linear guide arranged in front of the detector and may be operated by a stepper motor, the filter switch occurring rapidly and silently. The detector 20 is thus an optical detector and, in an embodiment, the transport of light occurs by means of an optical fiber.

In an embodiment, the detector 20 and the control unit C are configured in such a way that it is possible to perform readings within the same step at at least three different wavelength, so that the filter switch occurs rapidly.

In an embodiment, as shown in the figures, there are three reading stations (and therefore three detectors) for the execution of the ELISA test.

Stop Solution can be dispensed after the substrate and then the final reading (form example at 450 nm) is performed.

Obviously, any suitable detector may be used, configured to detect light at particular wavelengths.

The detector 30 apt to perform the CLIA test is, instead, a photomultiplier with bialkali photocathode sensitive in the range 400-650 nm. This photomultiplier is adapted to be suitably moved (in particular, it is able to perform two movements, one along the z axis—i.e. the vertical axis that is orthogonal to the working surface Fp—and one along the y axis or horizontal axis) to enable readings of different wells of the test device D.

In an embodiment, the detector 30 may be provided with a shutter to protect it from environmental light, as well as with a Peltier module to stabilize its temperature so as to keep the thermal noise as low as possible.

An ELISA reader optical check and calibration may be done at each start up. A CLIA reader check with dark reading and open shutter reading may also be performed and the signal range min max can be defined.

Therefore, summing up, an ELISA test may be performed, wherein, in its most simple form, antigens from the sample to be tested are attached to a surface. Then, a matching antibody is applied over the surface so it can bind the antigen. This antibody is linked to an enzyme and then any unbound antibodies are removed. In the final step, a substance containing the enzyme's substrate is added. If there is binding, the subsequent reaction produces a detectable signal, most commonly a color change, which is detected by the optical detector 20. In this case, chromogenic substrates such as TMB or ABTS may be used.

In the case of CLIA technique, the enzyme coupled to the detection antibody catalyzes a chemiluminescent reaction that results in the emission of photons producing light instead of a visible color change, and the detector 30 is therefore used. In this case, luminescent substrates such as luminol or acridinium ester may be used (whereas ELISA measures optical density, CLIA measures relative light unit).

In the case of the macroarray test, the image detector 40 may be, for example, a camera having high resolution (for example, from 8 MP to 13 MP) and adapted to allow the adjustment of the focus and of the exposure. In an embodiment, the camera is capable of moving in one direction (for example along the y axis, for example by the same movement means of the reader 30 for reading in CLIA), although other configurations are possible and fall within the scope of the present invention. The camera 40 is preferably configured to acquire a plurality of images (without limitation to a specific number) that are to be processed.

The macroarray test is advantageous, since it allows the determination of several analytes in a single test, with a single blood draw. As a consequence, it is very advantageous to integrate the macroarray test with the above-mentioned CLIA and ELISA tests. The test devices D are already configured also to allow said analysis, with the use of a specific substrate. Conveniently, for the macroarray test, the test device D is structurally identical to that of the other tests (possibly with a different color code), and one of the wells (for example, one of the wells D1) has a suitable grid (coated microplate) in which specific spots corresponding to specific reactions may appear; this grid further comprises also reference spots to orient the image and recognize which of the spots is associated to a given analyte. A method of detection by images in combination with a package of automated data analysis is thus implemented, thereby obtaining multi-parameter results within the same well, with small sample volumes, by means of acquisition of the image of the well, in particular of the grid, after the reaction has occurred, with the appearance of specific spots that are sensitive to respective reacting samples.

In other words, for the macroarray test, the reaction well D1 of the test device D comprises a coating, which has a matrix or grid that includes points of analysis (also called “spots”) which are related to specific biomarkers representing the measurement parameters of the macroarray test.

The measurement cycle may comprise the introduction of the diluted sample (for example, 1:100) into the reaction well D1, in which it is subjected to an incubation process for about twenty minutes, at the end of which, washing of said reaction well D1 is performed. Conjugated antibodies (for example, conjugated with peroxidase) are then added into the reaction well D1, after which an additional incubation and subsequent washing follow. The substrate for making the points of matrix on the bottom of the reaction well D1 visible is then supplied, and everything ends with a last washing. Finally, the image detector 40 acquires the image and the desired result is provided by processing said images.

Possibly, for the validation, the rotor may comprise suitable references between different housing seats.

In an embodiment, as mentioned above, the reader 40 may move along the axis Y and may be positioned on the CLIA reading module so as to use the same movement means, although other solutions are obviously possible.

In order to illuminate the spots lying on the bottom of the well of the test device D without generating light reflections, the macroarray reading unit comprises also a suitable illumination system which is suitably calibrated.

Furthermore, returning now to the mechanical structure of the apparatus 1, in order to transfer the sample to be analyzed and the reagents respectively in the pre-cycle step and during the execution of the test, the apparatus 1 comprises a set of needles (not shown in the figures).

In particular, the transfer of the samples occurs by means of a metal needle (indicated herein as “preparation needle”), for example a cannula made of stainless steel. This needle is provided with a crash sensor and with a capacitive level sensor. Obviously, also other materials, other configurations and other transferring means fall within the scope of the present invention.

In this embodiment, the preparation needle moves along the z axis by means of an appropriate linear actuator. In the case of other transferring devices, there may be other independent linear actuators, again for the movement along the z axis. Obviously, the movement of these actuators is calibrated so that the needle, during the descent, exerts a sufficient force to guarantee the correct puncturing of the film of the test device D. This needle is also apt to be moved also along the x axis, for example by means of a recirculating-ball linear guide, as well as along the y axis, by means of further linear guides.

After each time the preparation needle has performed drawing, it is then washed by internally and externally supplying a systemic solution by means of a pump. In an embodiment, this washing operation occurs in a dedicated well with four lateral jets, and specific means for drying the tip are provided.

There are then additional needles, in particular acting at the inner rotor 2′ for the preparation and the execution of the reaction. In particular, a dispensing unit with two needles for conjugate and substrate is provided, said needles being configured to move in parallel along the z axis along two independent linear guides and are driven by two stepper motors. A movement along the x axis, by means of a recirculating-ball linear guide, is also provided. As already previously observed for the pre-cycle step, also these needles may be provided with a capacitive level sensor. Also in this case, the needles may comprise a cannula made of stainless steel, on whose terminal part an extra polishing treatment may optionally be carried out. In particular, the terminal part of the needle has a beveled shape to facilitate the puncturing of the film. After each time these needles have performed drawing, they are internally and externally washed by supplying a systemic solution by means of a pump. In an embodiment, the washing wells comprise lateral jets.

There are then suitable stations for well washing, for example three independent washing stations, comprising washing and suction needles.

Moreover, the apparatus 1 may comprise a system for housing liquids (for example, washing buffer, cleaning solution and sanitizer), as well as a reservoir for liquid waste. As mentioned above, there are thus various bins, for example placed under the working surface Fp, with continuous monitoring of the levels (for example, by means of weight sensors) and with the possibility of both automatic and manual emptying of the reservoir for liquid waste.

Finally, the apparatus 1 comprises display means 50 in communication with the control unit C to display suitable user interfaces, for example user interfaces that show information relating to the performed test, as well as other information. For example, a display with a retro capacitive touch screen may be provided, as well as output means for the connection to a printer (for example, by means of a USB port). An internal printer for printing the measurement reports may also be provided.

As previously mentioned, the apparatus 1 comprises a memory unit MEM, for example a 64 GB or higher memory, to archive the database of the various tests. The database comprises a set of folders, in particular one folder for each session. Inside each folder, there are the different files containing the information about the sample, about the test device, about the methodology, and the raw data that generated the final result, as well as the images of the macroarray test.

Conveniently, there is a complete traceability of the results and the images obtained, with the possibility to access at any time the archive of the completed analyses, including displaying the kinetics (ELISA/CLIA) or the images of the macroarray test.

Summing up, the apparatus 1 of the present invention provides the execution of the following steps (obviously, this is only given by way of a non-limiting example of the present invention, wherein some steps may be eliminated/merged, other steps may be added, and other inverted):
manual loading of the racks with the samples;
identification of the samples by means of the barcode and reception of the list of the tests to be performed, for example from a host computer;
manual loading of the test devices D in the pre-cycle rotor 2″ through the dedicated door;
automatic transfer of the sample into the appropriate housing in the test device D;
execution of the pre-cycle on the test device D in the pre-cycle rotor 2″;
transfer of the test device D from the pre-cycle rotor 2″ to the reaction rotor 2′ for the execution of the analysis cycle;
automatic execution of all the operations provided by the test methodology to be carried out (shaking, transfers, washing and final reading). In this case, shaking is performed on the whole reaction rotor 2′ (i.e. identical for each test device). Shaking may be adjustable (i.e. the features of the vibration may be specific for the various tests), while the risk of creating incompatibility between the various tests is avoided by the presence of the pre-cycle rotor 2″, as also explained below. As mentioned, three metal needles are provided for transferring reagents and samples (one in the pre-cycle rotor 2″ for transferring the sample into the test device and two in the reaction rotor 2′); and
sending the result to LIS (“Laboratory Information System”) and subsequent automatic expulsion of the used test device into the appropriate discharging container.

In conclusion, the apparatus according to the invention achieves the set goals and has numerous advantages which have been already partially mentioned, thus brilliantly overcoming the technical problem and solving all the drawbacks of the prior art.

Advantageously, the apparatus of the present invention is capable of performing, even simultaneously, ELISA test, CLIA test and macroarray test, the latter with camera reading. There are thus three different reading systems that operate automatically and that may also operate simultaneously. The apparatus of the present invention is thus configured so that the three above-mentioned different technologies are carried out without creating incompatibility in the tests scheduling.

By means of the apparatus of the present invention, it is possible to carry out all the tests of interest by means of monotest kits (i.e. the above-mentioned test devices for a single determination-single sample test) that are already available and usable also on other instruments; the structure of these test devices is substantially the same for each type of test that can be performed on the apparatus of the present invention.

As described above, in addition to the tests carried out with ELISA technology, the apparatus according to the present invention allows to carry out analyses also by means of CLIA and macroarray methodologies, thereby allowing to extend and improve the analytical quality compared to performing only the ELISA test, by means of more innovative and/or complementary analyses. In particular, macroarray technology allows to perform multiple tests on a single device (which is useful, for example, in the allergology and autoimmunity field), whereas CLIA technology is superior to ELISA technology in terms of extension of the range of titration and of sensitivity, thereby obtaining a system with high versatility.

All this is obtained by means of a mechanical structure that is simple and compact. The ease of use and the wide choice of tests available on the market (infectious diseases, autoimmunity, endocrinology and tumor markers, allergy, bone metabolism, inflammation) make it the ideal tool to perform small-series diagnostic tests while ensuring the maximum flexibility in the organization of workflow of the clinical laboratory.

Conveniently, all the steps, from transferring the sample into the test device to sending the final result, are carried out in a controlled and automated way.

A further big advantage is the possibility of “continuous” loading the test devices, which allows to continuously load of more samples and reagents, even in the case in which an immediate analysis of the same is not possible. The user has only the task of positioning the test tubes and the test devices with the reagents into the appropriate housings (possibly paying attention to expose the respective barcode), the apparatus then providing for everything else. The functionality of continuous loading is always available; this guarantees, in addition to the above-mentioned versatility, also a great flexibility. In fact, the addition of the outer pre-cycle rotor, under ordinary working conditions, allows the user to immediately load possible samples to be analyzed at a later time even if they are incompatible with the ongoing analyses. In this way, the temperature and vibration incompatibility of some samples is limited, if not totally solved. Advantageously, the user may then interact with the apparatus when(s) he desires to introduce new tests; it is not necessary to wait the end of the ongoing analyses to be able to load new samples/devices.

Obviously, a person skilled in the art, in order to meet particular needs and specifications, may carry out several changes and modifications to the apparatus described above, all included in the protection scope of the invention as defined by the following claims.

1. An apparatus for performing immunometric tests, comprising:
at least one test device which is of the single use-type for a single sample test, wherein said test device comprises a body which houses, along a longitudinal axis thereof, a plurality of housings at least for a solid phase, for a sample to be analyzed, and reagents, wherein said housings comprise at least one reaction well containing the solid phase from a microtitration plate, at least one recess for the sample to be analyzed, and one or more recesses for respective one or more reagents;
a housing element configured to house the test device;
means acting on the housing element to allow the execution of the desired immunometric test; and
a control unit for managing the apparatus, wherein the apparatus comprises the following means for the automatic execution of different immunometric tests:
at least one detector configured to acquire signals and being in operative communication with the control unit which is configured to process said signals for the execution of Enzyme-Linked Immunosorbent Assay (ELISA) tests;
at least one detector configured to acquire signals and being in operative communication with the control unit which is configured to process said signals for the execution of Chemiluminescent Immunoassay (CLIA) tests; and
at least one image detector for acquiring images of the sample, said image detector being in communication with the control unit, and configured to process said images.

2. The apparatus according to claim 1, wherein the control unit is configured to perform said three immunometric tests individually or simultaneously.

3. The apparatus according to claim 1, wherein, based on the images of the image detector, the control unit is configured to perform macroarray tests, and wherein said control unit is configured to process the images by means of machine learning techniques.

4. The apparatus according to claim 1, wherein the detector for performing the ELISA test is a photometer.

5. The apparatus according to claim 1, wherein the detector for performing the CLIA test is a photomultiplier.

6. The apparatus according to claim 1, further comprising:
a housing area for test tubes containing the sample to be analyzed; and
a mechanical arm for taking the test tubes from the housing area and to perform the automatic transfer thereof into the test devices housed in the housing element.

7. The apparatus according to claim 1, wherein the housing element comprises a first rotor or inner rotor and a second rotor or outer rotor, which are arranged concentric one inside the other, wherein the second rotor is external to the first rotor and is a pre-cycle support adapted to house the test devices coming from a loading area, and wherein the first rotor is configured to receive the test devices from the second rotor and is the support in communication with the means for the execution of the reaction and of the desired test.

8. The apparatus according to claim 7, wherein the first rotor and the second rotor are moved by movement means which are independent from each other and controlled in an automated way, and wherein the second rotor is configured to perform a pre-cycle in which the sample in the test tube is transferred into the appropriate housing of the test device, and the first rotor is configured to perform a shaking of the test device containing the sample and reagents.

9. The apparatus according to claim 8, further comprising one or more needles for transferring the samples and reagents in the pre-cycle step in the second rotor and for carrying out the immunometric test in the first rotor.
